(19) United States
(12) Patent Application Publication
(10) Pub. No.: US 2026/0275328 A1
(43) Pub. Date: Sep. 17, 2026

(54) NON-TOXIC FORMULATION FOR COLLECTING BIOLOGICAL SAMPLES, AND DEVICE FOR CAPTURING AND ELUTING NUCLEIC ACIDS IN THE SAMPLES

(71) Applicant: Definitive Biotechnologies LLC, Elkridge, MD (US)

(72) Inventors: Peter Latham, Acton, MA (US); Timothy Alcorn, Raleigh, NC (US); Todd Haran, Bloomfield, NY (US); Steve Rapp, Rochester, NY (US); Jones Haran, West Bloomfied, NY (US); Scott Rosebrough, Avon, NY (US)

(21) Appl. No.: 19/674,769

(22) Filed: May 12, 2026

Related U.S. Application Data
(62) Division of application No. 18/176,949, filed on Mar. 1, 2023, now abandoned.
(60) Provisional application No. 63/268,742, filed on Mar. 1, 2022.

(51) Int. Cl. C12N 15/10

(52) U.S. Cl. CPC C12N 15/1017

(57) ABSTRACT

A formulation for collecting a biological sample of saliva or nasal fluid and capturing nucleic acids in the collected sample has non-toxic chaotropic agents, ethanol, and coloring and/or flavoring agents. The formulation is receivable within an oral cavity or nasal cavity to collect the sample and the non-toxic chaotropic agent(s) lyse the sample cells. A device has a sample port for receiving the sample-containing formulation and a solid-state membrane in fluid communication with the sample port. A first pump causes the sample-containing formulation to flow across the solid-state membrane and into a waste chamber. The ethanol binds nucleic acids in the lysed cells of the sample to the solid-state membrane. A second pump causes the eluent to flow from an eluent chamber across the membrane, elute captured nucleic acids from the membrane, and flow with the captured nucleic acids into an eluent reservoir.

### CROSS-REFERENCE TO RELATED PATENT APPLICATION

This patent application is a divisional of co-pending U.S. patent application Ser. No. 18/176,949, filed Mar. 1, 2023, entitled “Non-Toxic Formulation For Collecting Biological Samples, And Device For Capturing And Eluting Nucleic Acids In The Samples,” which claims priority under 35 U.S.C. § 119(e) to U.S. provisional patent application No. 63/268,742, filed Mar. 1, 2022, also entitled “Non-Toxic Formulation For Collecting Biological Samples, And Device For Capturing And Eluting Nucleic Acids In The Samples,” each of which is hereby incorporated by reference in its entirety as part of the present disclosure.

### FIELD OF THE INVENTION

The present invention relates to devices for and methods of isolating, concentrating, amplifying, and detecting nucleic acids in biological samples, such as saliva or mucus, and more particularly, to the collection of oral cavity or nasal cavity samples, compositions that contain non-toxic sample preparation materials to collect and prepare the samples for capturing nucleic acids therein, and for capturing nucleic acids in biological samples and amplifying the captured nucleic acids in reaction chambers.

### BACKGROUND INFORMATION

Saliva is an attractive sample type for clinical diagnostics because it is non-invasive and can be used to detect many diagnostic targets that are traditionally collected via nasal swabs, sputum or blood samples. However, saliva poses several issues for use in microfluidic devices for molecular diagnostics (diagnostics based on RNA or DNA targets). First, saliva can vary significantly in viscosity which can affect the fluid dynamics of a device. Second, it is difficult to standardize the volume of saliva collected and used because of imprecise sample collection methods and the differential amounts of saliva an individual can produce and spit. In addition, saliva can foam thus introducing bubbles into microfluidic channels that can disrupt the flow of fluids in a device.

To solve some of these issues, the sample preparation of saliva (cell lysis) is typically done external to the device or microfluidic system where the user adds saliva to a container external to the system that contains sample preparation chemicals. This can help normalize the viscosity of the sample before introduction into the device and can help address foaming issues, but it does not address the differential volume of saliva collected and can expose the user to potentially toxic sample preparation chemicals.

It is an object of the present invention, and/or of the currently preferred embodiments thereof, to overcome one or more of the above-described drawbacks and/or disadvantages of the prior art.

### SUMMARY OF THE INVENTION

In accordance with a first aspect, the present invention is directed to a device comprising (i) a sample port or chamber for receiving therein a sample-containing mixture containing therein a biological sample; (ii) a solid-state membrane in fluid communication with the sample port or chamber and configured to receive the sample-containing mixture therefrom, and allow the sample-containing mixture to pass across the membrane and capture nucleic acids in the biological sample on the membrane; (iii) a first pump in fluid communication the solid-state membrane and a waste chamber in fluid communication with the pump, wherein actuation of the first pump causes the sample-containing mixture to flow across the solid-state membrane and into the waste chamber; (iv) an eluent chamber containing an eluent therein; (v) an eluent reservoir in fluid communication with the solid-state membrane; and (vi) a second pump in fluid communication with the solid-state membrane and the eluent chamber, wherein actuation of the second pump causes the eluent to flow from the eluent chamber across the solid-state membrane, elute captured nucleic acids from the solid-state membrane, and flow with the captured nucleic acids into the eluent reservoir.

In some embodiments of the present invention, the first pump is a syringe containing a barrel and a plunger received within the barrel. The barrel defines the waste chamber therein. Movement of the plunger either draws or pulls, or pushes the sample-containing mixture across the solid-state membrane and into the waste chamber of the barrel.

In some embodiments of the present invention, the second pump is movable between a non-actuated position and an actuated position. The eluent chamber includes a frangible or breakable wall that is breakable by movement of the second pump between the non-actuated position and the actuated position to pump eluent from eluent chamber across the solid-state membrane and into the eluent reservoir. In some such embodiments, the second pump is a plunger. Movement of the plunger from the non-actuated position to the actuated position breaks the frangible or breakable wall of the eluent chamber and pushes or pulls the eluent across the solid-state membrane and into the eluent reservoir. In other embodiments, the second pump is a syringe including a barrel and a plunger received within the barrel. Movement of the plunger pushes or pulls the eluent across the solid-state membrane and into the eluent reservoir.

Some embodiments of the present invention further comprise a first valve in fluid communication between the sample port or chamber and the solid-state membrane and configured to allow the sample-containing mixture to flow in the direction from the sample port or chamber to the solid-state membrane, but prevent liquid flow in the opposite direction.

Some embodiments of the present invention further comprise a second valve in fluid communication between the solid-state member and the eluent reservoir and configured to allow fluid flow in the direction form the solid-state member into the eluent reservoir, but prevent liquid flow in the opposite direction.

Some embodiments of the present invention further comprise at least one reaction chamber and at least one conduit, such as a capillary conduit, in fluid communication between the eluent reservoir and the reaction chamber. The capillary conduit(s) is (are) configured to allow the eluent with captured nucleic acids to flow by capillary action through the capillary conduit(s) and into the reaction chamber(s).

In accordance with another aspect, the present invention is directed to a device comprising: (i) first means for receiving therein a sample-containing mixture containing therein a biological sample; (ii) second means in fluid communication with the first means for receiving the sample-containing mixture therefrom, for allowing the sample-containing mixture to pass across the second means, and for capturing nucleic acids in the biological sample on the second means; (iii) third means for receiving and holding the sample-containing mixture after passing across the second means; (iv) fourth means in fluid communication with the second means and the third means for pumping the sample-containing mixture across the second means and into the third means; (v) fifth means for containing an eluent therein and for allowing the eluent to flow across the second means after the sample-containing mixture passes across the second means, and for removing from the second means captured nucleic acids from the biological sample with the eluent; (vi) sixth means in fluid communication with the solid-state membrane for receiving and collecting the eluent with captured nucleic acids from the biological sample therein; and (vii) seventh means in fluid communication with the second means and the fifth means for pumping the eluent to flow from the fifth means across the second means, elute captured nucleic acids from the second means, and flow with the captured nucleic acids into the sixth means.

In some embodiments of the present invention, the first means is a sample port or chamber, the second means is a solid-state membrane, the third means is a waste chamber, the fourth means is a first pump, the fifth means is an eluent chamber, the sixth means is an eluent reservoir, and the seventh means is a second pump.

In accordance with another aspect, the present invention is directed to a formulation for collecting a biological sample of saliva or nasal fluid and capturing nucleic acids in the collected biological sample on a solid-state membrane. The formulation comprises: (i) one or more non-toxic chaotropic agents; (ii) ethanol; and (iii) coloring and/or flavoring agents. The formulation is receivable within an oral cavity or a nasal cavity to collect the biological sample of saliva or nasal fluid therefrom. The one or more non-toxic chaotropic agents lyse the cells of the biological sample, if necessary, and the ethanol binds nucleic acids in the lysed cells of the biological sample to the solid-state membrane.

Some embodiments of the present invention comprise about 0.1% to about 40% w/v non-toxic chaotropic agents and about 5% to about 30% w/v ethanol. In some such embodiments, the non-toxic chaotropic agents are selected from the group including the following individually or in any combination thereof: (i) about 5% to about 30% w/v urea; about 0.1% to about 3% w/v sodium lauryl sulfate; and about 2% to about 40% w/v ammonium trichloroacetate.

In some embodiments of the present invention, the formulation is provided in combination with a long-chain fatty alcohol wash configured to flow over the solid-state membrane following the formulation to substantially eliminate any residual ethanol of the formulation on the solid-state membrane.

One advantage of the present invention, and/or of embodiments thereof, is that it can provide a solution to the saliva collection issues encountered in the above-described prior art. Another advantage is that the system allows the use of a mouthwash or nasal wash that a user can swish in the mouth or spray into the nose before introduction of the saliva or nasal fluid into the microfluidic or other nucleic acid isolation and purification system. Yet another advantage is that the mouthwash or nasal wash contains sample preparation chemicals that are non-toxic thus allowing partial and/or complete sample preparation (e.g., cell lysis) before introduction into the microfluidic or other nucleic acid isolation and purification system or into an external sample collection cup. A further advantage of the invention and/or of embodiments thereof is that they can normalize the volume of sample collected, eliminate or substantially eliminate issues of viscosity and foaming, and eliminate, substantially eliminate or minimize the exposure of users to toxic chemicals. Yet another advantage is that the invention can be extended to a nasal wash for easier collection of nasal samples.

Other objects and advantages of the present invention, and/or of embodiments thereof, will become more readily apparent in view of the following detailed description of embodiments and accompanying drawings.

### BRIEF DESCRIPTION OF THE DRAWINGS

FIG. 1A is a somewhat schematic illustration of an embodiment of a microfluidic system of the present invention indicated generally by the reference number 10 comprising a microfluidic device 12 for receiving through an inlet port 14 a sample mixture 16. The sample mixture 16 contains a biological sample, such as saliva or nasal fluid (e.g., mucus), non-toxic chaotropic agents for cell lysis, ethanol and coloring and/or flavoring agents. The sample mixture 16 is introduced into the inlet port 14 of the microfluidic device 12 by a transfer device, such as from a syringe 18A, funnel 18B or collection vessel 18C, and through a sample introduction tube 18D. The inlet port 14 is in fluid communication with a capture membrane 20 (e.g., a glass or solid-state membrane), and a syringe or other pump 22 is actuated to draw or pull the sample mixture 16 across or through the solid-state membrane 20 where RNA and/or DNA in the sample is captured by and collected on the membrane and the remainder of the sample mixture is pulled into the syringe 22 as waste. An elution blister or other pump 24 containing eluent 26 (e.g., water (lined with the color blue)) is then depressed or actuated to break the blister and release and pump the eluent across the solid-state membrane 20 to remove the captured RNA and/or DNA on the membrane and carry the captured RNA and/or DNA into an eluent reservoir 28 where the eluent with captured RNA and/or DNA is allowed to collect or pool. A plurality of capillary tubes 30, 30 are connected in fluid communication between the reservoir 28 and a plurality of reaction chambers 32, 32 and a negative control chamber 40, and are configured to transfer the eluent with captured RNA and/or DNA by capillary action from the reservoir into the reaction chambers and negative control chamber.

FIG. 1B are somewhat schematic illustrations of the syringe 18A, funnel 18B and sample collection vessel 18C of FIG. 1A, each connected or connectable in fluid communication with the sample introduction tube 18D preferably by a lockable or locking connector 50.

FIG. 2 is a somewhat schematic illustration of another embodiment of the microfluidic system 10 of FIG. 1 where the microfluidic device 12 includes an elution syringe or other pump 24′ prefilled with eluent 26 (e.g., water (lined for the color blue)) instead of the elution blister 24 of FIG. 1. The elution syringe 24′ is actuated to release and pump the eluent 26 across the solid-state membrane 20 to remove the captured RNA and/or DNA on the membrane and carry the captured RNA and/or DNA into the eluent reservoir 28 where the eluent 26 with captured RNA and/or DNA is allowed to collect or pool prior to transfer by capillary action into the reaction chambers, 32, 32.

FIG. 3 is a somewhat schematic illustration of the microfluidic system 10 of FIG. 1 highlighting in the rectangle 34 the features or portions of the system focused on sample collection, RNA/DNA isolation and purification.

FIG. 4 is a somewhat schematic illustration of the microfluidic system 10 of FIG. 1 showing a first step of use of the device 12 where a user collects a biological sample and introduces a sample mixture 16 into the microfluidic device in accordance with any of three options 18A, 18B or 18C.

FIG. 5 is a somewhat schematic illustration of the microfluidic system 10 of FIG. 1 showing a second step of use of the device 10 where a patient or other user pulls the empty syringe 22 of the microfluidic device 10 to, in turn, pull the sample mixture 16 (lined for the color green) across the capture membrane 20 and into the barrel of the syringe 22 which acts as waste container.

FIG. 6 is a somewhat schematic illustration of the microfluidic system 10 of FIG. 1 showing a third step of use of the device 12 where the patient or other user continues to pull the plunger of the syringe 22 of the device 10 which either pulls dodecanol (or other long-chain fatty alcohol) 36 (lined for the color red) across the membrane 20 and/or air across the membrane 20 to remove substantially all residual ethanol from the membrane.

FIG. 7A is a somewhat schematic illustration of the microfluidic system 10 of FIG. 1 showing a fourth step of use of the device 12 where a patient or other user depresses the elution blister 24 which pushes the elution buffer 26 across the membrane 20 to elute the captured DNA and RNA off the membrane 20 and into the capture reservoir 28 (where the eluent with captured DNA and RNA is lined for the color blue). Fluid flow is controlled by one-way valves 38, 38 (shown as arrows lined for the color red) (i) in fluid communication between the inlet port 14 and the inlet side of the membrane 20, and (ii) in fluid communication between the outlet side of the membrane 20 and the capture reservoir 28. The valves 38, 38 prevent liquid from flowing backwards from the capture reservoir 28 and/or reaction chambers 32, 32 into the membrane 20, and prevent liquid and/or other material from flowing backwards through the inlet port 14 and into the transfer/collection device(s) 18A, 18B, 18C and/or 18D. Once the captured RNA and DNA is in the capture reservoir 28, the reaction chambers 32, 32 are filled via capillary action by the capillary conduits 30, 30 extending in fluid communication between the capture reservoir 28 and each respective reaction chamber 32, 32 and a negative control chamber 40, where the eluent dissolves the reaction chemistry pre-stored in the reaction chambers as lyophilized beads. Depression of the elution blister 24 also activates a device heating element 42 in thermal communication with the reaction chambers 32, 32 and negative control chamber 40 for heating the chambers to the required temperature for the reaction.

FIG. 7B is cross-sectional view of the syringe barrel of the device of FIG. 7A showing the collected waste dodecanol 36 (lined for the color red) on top of the waste sample mixture 16 (lined for the color green) within the barrel of the syringe 22.

FIG. 8 is a somewhat schematic illustration of the microfluidic system 10 of FIG. 2 showing the fourth step of use of the device 12 where a patient or other user depresses or otherwise actuates the elution syringe 24′ which pushes the elution buffer 26 across the membrane 20 to elute the captured DNA and RNA off the membrane and into the capture reservoir 28 (where the elution buffer with captured DNA and RNA is lined for the color blue). Fluid flow is controlled by the one-way valves 38, 38 (shown as arrows lined for the color red) (i) in fluid communication between the inlet port 14 and the inlet side of the membrane 20, and (ii) in fluid communication between the outlet side of the membrane 20 and the capture reservoir 28. The valves 38, 38 prevent fluid from flowing backwards from the capture reservoir 28 and/or reaction chambers 32, 32 into the membrane 20, and prevent any material from flowing backwards through the inlet port 14 into the transfer/collection device(s) 18A, 18B, 18C and/or 18D. Once the RNA and DNA is in the capture reservoir 28, the reaction chambers 32, 32 and negative control chamber 40 are filled via capillary action through the capillary conduits 30, 30 which dissolves the reaction chemistry pre-stored in the chambers as lyophilized beads. In FIG. 8, the pre-stored reaction chemistry is shown as a circle within each empty reaction chamber 32, 32, whereas in FIG. 9 below, the reaction chemistry is not shown because at that stage it is dissolved in the elution buffer with captured DNA and RNA. Depression of the plunger of the elution syringe 24′ also activates the device 10 heating element 42 for heating the reaction chambers 32, 32 and negative control chamber 40 to the required temperature for the reaction.

FIG. 9 is a somewhat schematic illustration of the microfluidic system 10 of FIG. 1 showing a fifth step of use of the device 12 where the reaction chambers 32, 32 are heated by the heating element 42 of the device to initiate an amplification reaction where the target RNA/DNA is amplified, if present. Positive amplification is detected via user visualization, such as color change, through transparent/translucent windows 44, 44 of the reaction chambers 32, 32. For example, as shown in FIG. 9, a positive amplification in each reaction chamber 32, 32 is lined for the color green and is visible through transparent/translucent windows 44, 44 of the reaction chambers 32, 32, whereas the negative control in the negative control chamber 40 is lined for the color blue and is visible through the negative control chamber transparent/translucent window 44. As may be recognized by those of ordinary skill in the pertinent art based on the teachings herein, positive amplification may be detected in any of numerous different ways that are currently known, or that later become known, such as by fluorescence, a change in turbidity, or by digital image capture via an imbedded CCD camera or other sensor, or an external device such as a smart phone.

FIG. 10 is a perspective view of another device embodying the present invention and indicated generally by the reference numeral 112A for sample collection, RNA/DNA isolation and purification.

FIG. 11 is a side elevational view of the device of FIG. 10 where a lower manifold 152 thereof is shown in broken lines to illustrate the interior thereof.

FIG. 12 is a top plan view of a multiplex amplification chip or device 112B that is usable with the device 112A above for amplifying the RNA/DNA that is isolated and purified in the device 112A and for detecting the targeted RNA/DNA therein.

FIG. 13A is another top plan view of the multiplex amplification device 112B of FIG. 12 showing in cross-hatched lines a single vent membrane component 149 covering a plurality of vented ports 148, 148 in fluid communication with respective reaction chambers 132, 132 and the negative control chamber 140 for venting the chambers therethrough.

FIG. 13B is another top plan view of the multiplex amplification device 112B of FIG. 12 showing in cross-hatched lines a one-piece optically clear material 144 covering each reaction chamber 132, 132 and the negative control chamber 140 to thereby form a transparent/translucent viewing window over each reaction chamber or negative control chamber for viewing the interior of each chamber therethrough.

FIG. 14 is a cross-sectional view of the of the multiplex amplification device 112B of FIG. 12 prior to heating and initiation of an amplification reaction.

FIG. 15 is another cross-sectional view of the of the multiplex amplification device 112B of FIG. 12 prior to heating and initiation of an amplification reaction, and showing the pre-stored lyo-bead or other reaction chemistry in one of the reaction chambers 132, 132, and a wax pellet 172 located in the respective fluid conduit 130 in fluid communication between the eluent reservoir 128 and the respective reaction chamber 132 to prevent fluid communication therethrough prior to heating for the amplification reaction.

FIG. 16 is a cross-sectional view of the of the multiplex amplification device 112B of FIG. 12 showing the device after amplification when the lyo-bead or other reaction chemistry has been dissolved and the wax pellet melted away, and including a heater 142, such as an electric or chemical heater, in thermal communication with the reaction chambers 132, 132, and an excitation light emitting diode (“LED”) 174 or other light source that causes the positive indicator to fluoresce, such that when viewed by an eye 176 of an operator, the fluorescence indicates positive detection of target DNA/RNA.

FIG. 17 is a cross-sectional view another version of the of the multiplex amplification device 112B of FIG. 12 showing the device after amplification when the lyo-bead or other reaction chemistry has been dissolved and the wax pellet melted away, and including an optical sensor 174 configured to measure emission wavelength and intensity (e.g., color and/or power) within the reaction chambers 132, 132 to sense a positive detection of target DNA/RNA and transmit signals to, for example, a smart phone or other display (not shown), indicative thereof.

### DETAILED DESCRIPTION

In a currently preferred embodiment, the sample collection formulation 16 is provided in the form of a mouthwash or a nasal spray or flush. The sample collection formulation 16 contains the following components:
1) Non-toxic chaotropic agents for cell lysis used individually or in combination. Such agents include but are not limited to urea which is used in artificial saliva; sodium lauryl sulfate which is used in toothpaste; and/or ammonium trichloroacetate which is used to treat lesions on the skin and mucus membranes. The amount of urea is preferably within the range of about 5% to about 30% w/v, is more preferably within the range of about 10% to about 25% w/v, and is even more preferably within the range of about 15% to about 20% w/v. The amount of sodium lauryl sulfate is preferably within the range of about 0.1% to about 3% w/v, is more preferably within the range of about 0.2% to about 1.5% w/v, and is even more preferably within the range of about 0.4% to about 0.7% w/v. The amount of ammonium trichloroacetate is preferably within the range of about 2% to about 40% w/v, is more preferably within the range of about 4% to about 20% w/v, and is even more preferably within the range of about 8% to about 10% w/v. As may be recognized by those of ordinary skill in the pertinent art based on the teachings herein, some applications may not require cell lysis, such as where there is free-floating nucleic acids. However, such applications may nevertheless require the non-toxic chaotropic agents to chaotrope the cells and facilitate binding nucleic acids to the solid-state membrane or other nucleic acid capture device.
2) Ethanol to facilitate capture of RNA and DNA. The amount of ethanol is preferably within the range of about 5% to about 30% w/v, is more preferably within the range of about 10% to about 25% w/v, and is even more preferably within the range of about 15% to about 20% w/v.
3) Coloring and/or flavoring agents.

The following is a representative formulation:
1) about 20% w/v ethanol;
2) about 0.1% w/v sodium lauryl sulfate;
3) about 20% w/v ammonium trichloroacetate;
4) about 0.042% w/v menthol; and
5) water.

The device and method also can employ a long-chain fatty alcohol wash. Ethanol is an inhibitor of many amplification reactions. In the system disclosed herein, ethanol is used to bind nucleic acids to the glass or solid-state membrane 20 surfaces. Residual ethanol on the glass or solid-state membrane surface or in the fluidic channels can be carried into the reaction chambers 32, 32 when nucleic acids are eluted off the glass or solid-state membrane surfaces. To solve this issue, the fluidic channels and glass surfaces can be washed with a long chain (>4) fatty alcohol, such as 2-dodecanol, that is clear and hydrophobic. This provides several advantages: a) the fatty alcohol displaces and solubilizes residual ethanol in the system; b) residual fatty alcohol does not inhibit amplification reactions or resulting visualization; and c) residual fatty alcohol can be used to provide a barrier to evaporation of water from the reaction solutions. Other long chain fatty alcohols that can be used include but are not limited to the following (used individually or in any combination):
1) Dodecanol;
2) Octanol;
3) Stearyl Alcohol;
4) Lauryl Alcohol;
5) Cetyl Alcohol;
6) Oleyl Alcohol; and
7) Butyl Alcohol.

There are several options for using the above formulation as a mouthwash or nasal spray or wash, including the following:

Option 1: The mouthwash/nasal spray or wash is coated onto a gauze or other wad of absorbent material that is used either to swab the mouth or nose. The swab is then compressed by a syringe 18A in the inlet 14 of the microfluidic device 12 to release the materials therefrom and into the device. The syringe 18A may also contain 2-dodecanol 36 (about 50 to about 500 μl (or other long-chain fatty alcohol, as indicated above) for improved assay performance. The 2-dodecanol or other long-chain fatty alcohol(s) 36 sits in a chamber of the syringe 18A such that it is introduced into the device 12 following the saliva mixed with the mouthwash/nasal spray.

Option 2 (saliva only): The user swishes the mouthwash in the mouth and then spits the mouthwash with saliva into the microfluidic device with the help of a funnel 18B or like device.

Option 3 (saliva only): The user swishes the mouthwash in the mouth and then spits the mouthwash with saliva into a secondary collection vessel or cup 18C which is pre-loaded with additional chaotropic agents and/or ethanol 46 that are released before, upon or after sealing the cup. For example, the collection vessel 18C can include a chamber with a frangible or breakable wall containing therein the additional chaotropic agents and/or ethanol 46. The vessel closure may include a piercing member such that upon closing the vessel 18C with the closure, the piercing member breaks the wall to thereby allow mixture of the mouthwash and saliva with the additional chaotropic agents and/or ethanol 46 within the vessel. The user then agitates the cup 18C (e.g., by shaking it) to contain a lyse and chaotrope sample mixture within the vessel, and then introduces the mixture from the vessel into the inlet 14 of the microfluidic device 12 via, for example, another transfer device, such as a syringe 18A, which may also contain 2-dodecanol (about 50 to about 500 μl (or other long-chain fatty alcohol) 36 for improved assay performance. The 2-dodecanol 36 may sit in a chamber of the syringe 18A such that it is introduced into the device 12 following the saliva-containing mixture.

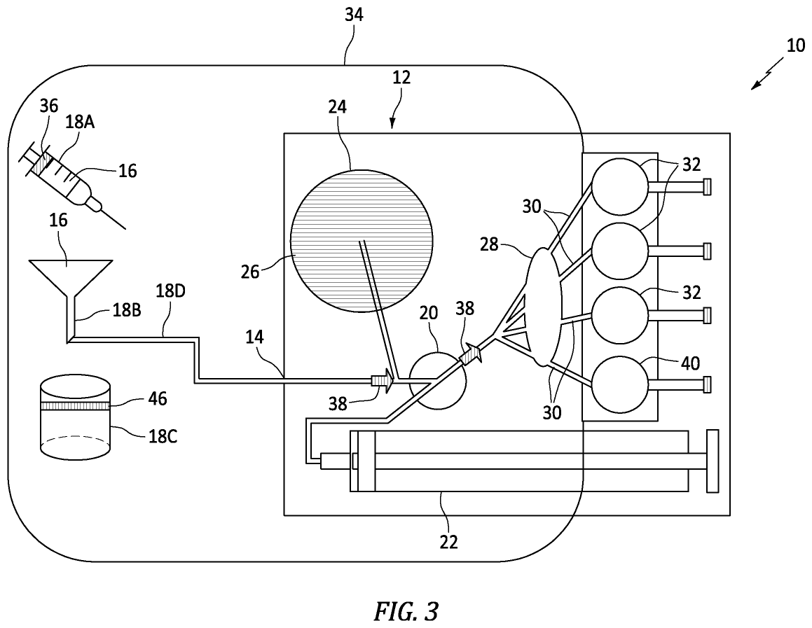

As shown typically in FIG. 3, the following are the principal or key components of the device 10. The following such components are located outside of the microfluidic device 12:
1) a sample collection formulation or solution 16 (e.g., the mouthwash/nasal spray or wash);
2) a collection device (e.g., a collection cup or vessel 18C, or a swab located at the distal end of a syringe 18A for absorbing the saliva therein, and releasing the saliva when compressed upon actuation of the syringe); and
3) a transfer device (e.g., a syringe 18A or funnel 18B).

The following such components are located on or within the microfluidic device 12:
1) A tube/channel 18D that connects the microfluidic device 12 to the transfer device 18A, 18B and/or 18C;
2) A DNA/RNA capture membrane (or solid-state membrane) 20. In the illustrated embodiment of the solid-state membrane, the inlet or inlet side of the membrane is on the top, and the outlet or outlet side of the membrane is on the bottom.
3) A syringe 22 that “pulls” materials from the transfer device/inlet port 14, across the solid-state membrane 20, and into the syringe 22 (defining a waste chamber therein);
4) One-way valves 38, 38 that control the flows of fluids into and across the solid-state membrane 20, and from the solid-state membrane 20 to either the waste chamber in the syringe 22 or to the reservoir 28 for capillary transfer to the reaction chamber(s) 32, 32;
5) A blister mechanism 24, or syringe 24′ or other pump/dispensing device, pre-loaded with an elution buffer 26 (e.g., water). Although standard pull-type syringes 22, 24′ are shown, each syringe may be a reverse pressure syringe, or other type of syringe or like pumping/dispensing device that is currently known, or that later becomes known.
6) A reservoir 28 that pools the elution 26 before loading same into the reaction chambers 32, 32 such that fluids flow from the solid-state membrane 20 into the reservoir 28 and pool within the reservoir before the elution flows into the reaction chamber(s) 32, 32. One advantage of the reservoir 28 is that it can be used to pool the purified RNA/DNA to facilitate obtaining a substantially consistent target concentration, including, for example, across multiple reaction chambers;
7) Capillary tubes or conduits 30, 30 that transfer the elution from the reservoir 28 into the reaction chambers 32, 32 and negative control chamber 40. One advantage of using capillary tubes is that they provide a capillary fill action into the reaction chamber(s) which, in turn, facilitates bubble management;
8) Reaction chambers 32, 32 pre-loaded with lyophilized amplification reagents and a negative control chamber 40;
9) A heater 42 for the amplification reaction; and
10) Gas permeable membranes (i.e., gas only valves) 48, 48 at the distal ends of the reaction chambers 32, 32 and negative control chamber 40 to allow for air to escape and liquid to fill the respective chambers. As shown in the illustrated embodiment, a plurality of venting lines extend in fluid communication between the reaction chambers 32, 32 and negative control chamber 40 and respective gas only valves 48, 48.

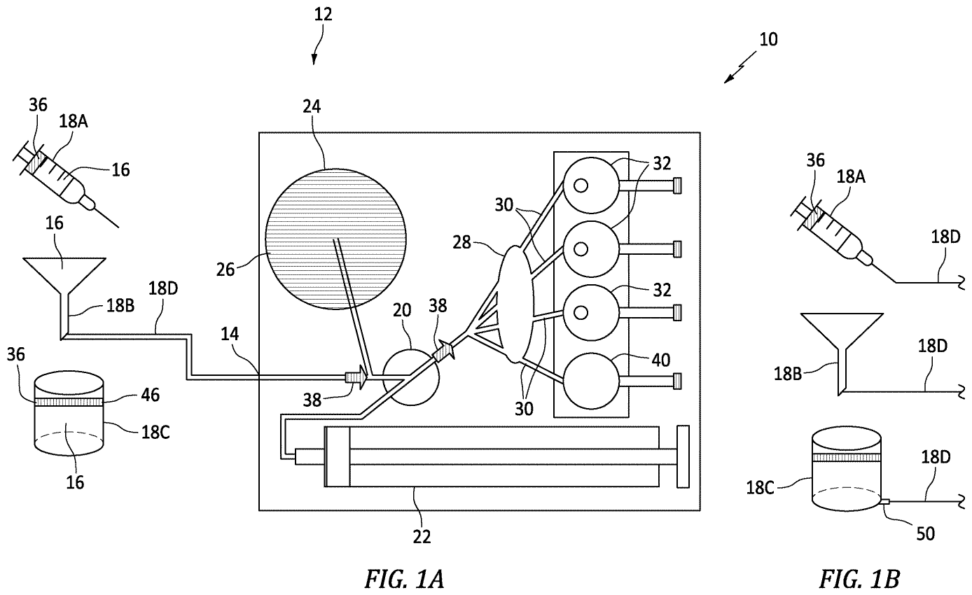

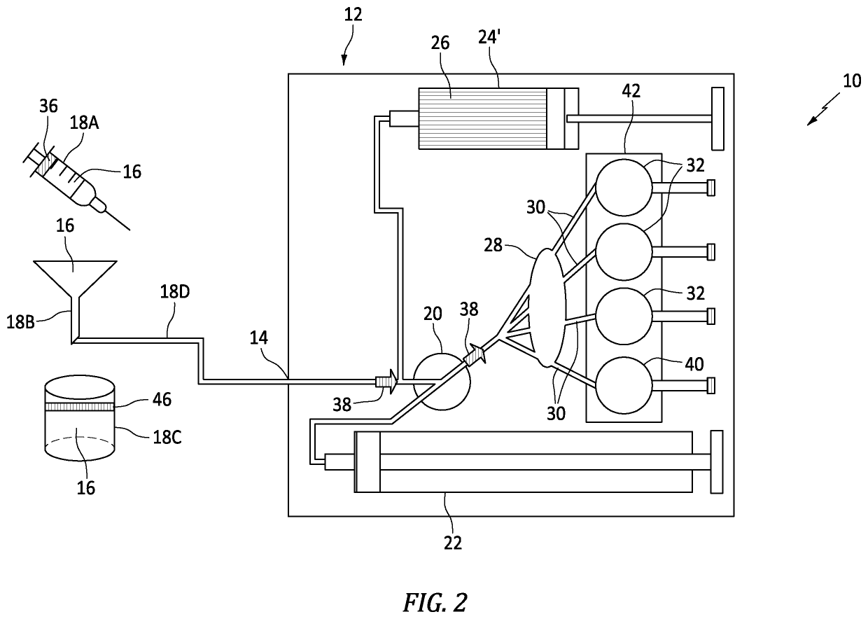

In the operation of the microfluidic system 10, and with reference to FIGS. 1A, 1B and 2, the sample-containing mixture 16 is introduced into the device 12 using options 18A, 18B or 18C and the elution buffer 26 (e.g., water) in encased or contained within the elution blister 24 (FIG. 1A) or the pre-filled elution syringe 24′ (FIG. 2). Three options are illustrated for sample collection and introduction into the device 12. Using option 18A, a collection and transfer syringe is used. If dodecanol 36 is needed, the dodecanol is contained within a sealed section of the syringe 18A and is introduced into the device 12 once the sample is introduced by a lockable or locking mechanism between the syringe and the introduction tube 18D (e.g., a flexible tube). Using option 18B, a funnel is connected to the device 12 via a lockable or locking flexible tube 18D. As can be seen, dodecanol is not used in this particular embodiment. The funnel 18B preferably connects directly to the introduction tube 18D via a locking mechanism of a type known to those of ordinary skill in the pertinent art. Using option 18C, if dodecanol is needed, a chamber within the sample collection vessel 18C contains and releases dodecanol 36 when the vessel is closed. The mixed contents of the sample collection chamber may be “pulled” into the device via the syringe 18A, if desired. The connection between the collection cup 18C and inlet port 14 of the device 12 is a flexible tube 18D having a lockable or locking connection 50 (FIG. 1B) of a type known to those of ordinary skill in the pertinent art.

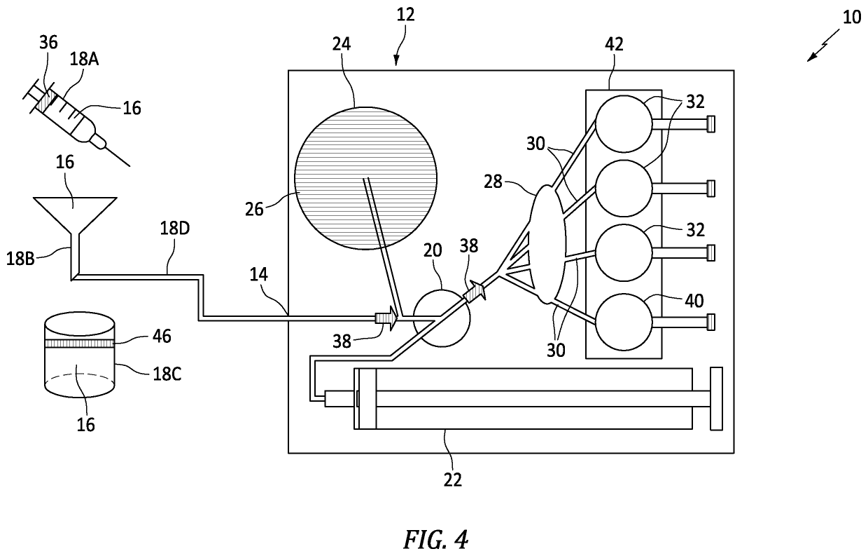

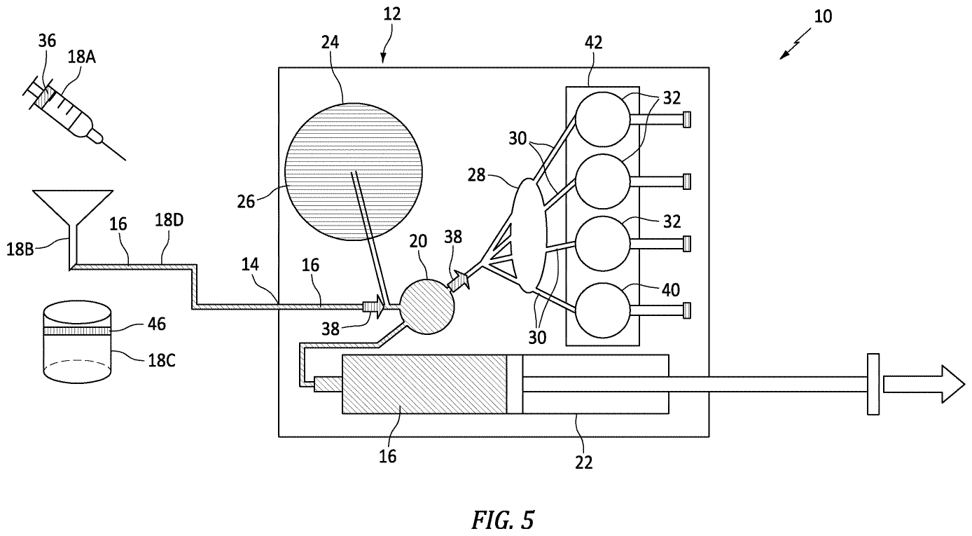

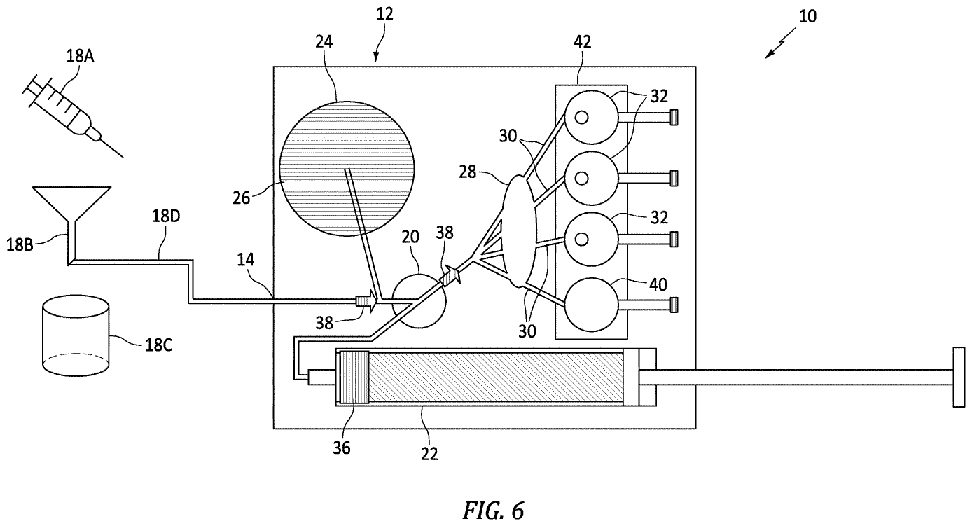

As shown in FIG. 4, a first step of using the device 12 includes the patient or other user collecting a biological sample 16 and introducing the sample mixture 16 into the microfluidic device 12 in accordance with any of options 18A, 18B or 18C described above. As indicated by the arrow in FIG. 5, in the second step, the patient or other user pulls the plunger of the empty syringe 22 which in turn pulls the sample mixture 16 across the capture membrane 20 and into the barrel of the syringe, which acts as waste container. As shown in FIG. 6, in the third step, the patient or other user continues to pull the plunger of the syringe 22 until the plunger hits a stop in the syringe which, in turn, pulls dodecanol 36 across the membrane 20 to remove all residual ethanol. In another embodiment, there is no need for dodecanal, and in the third step, the syringe 22 pulls air across the membrane 20 such that residual air (e.g., residual air in the syringe 18A, funnel 18B, or collection cup 18C) eliminates the ethanol.

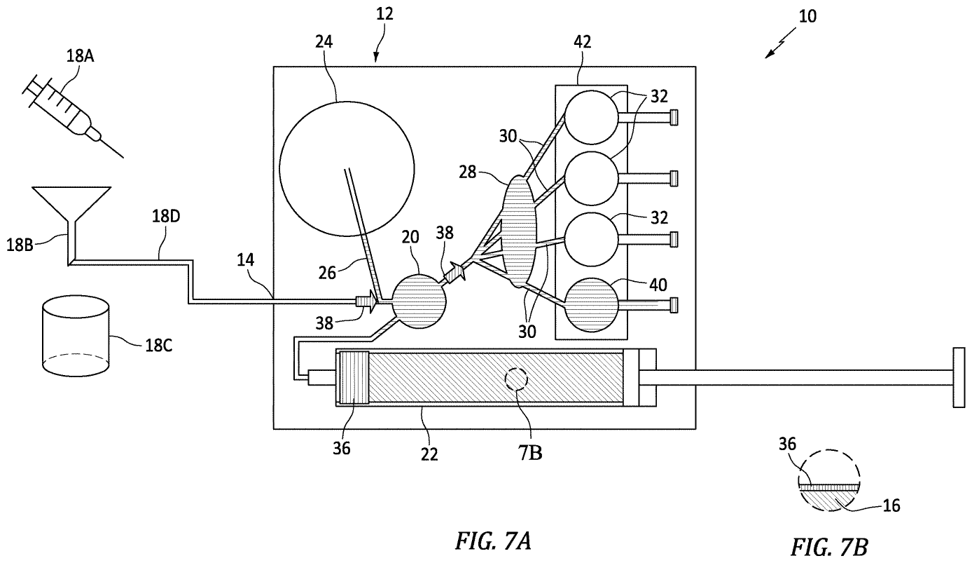

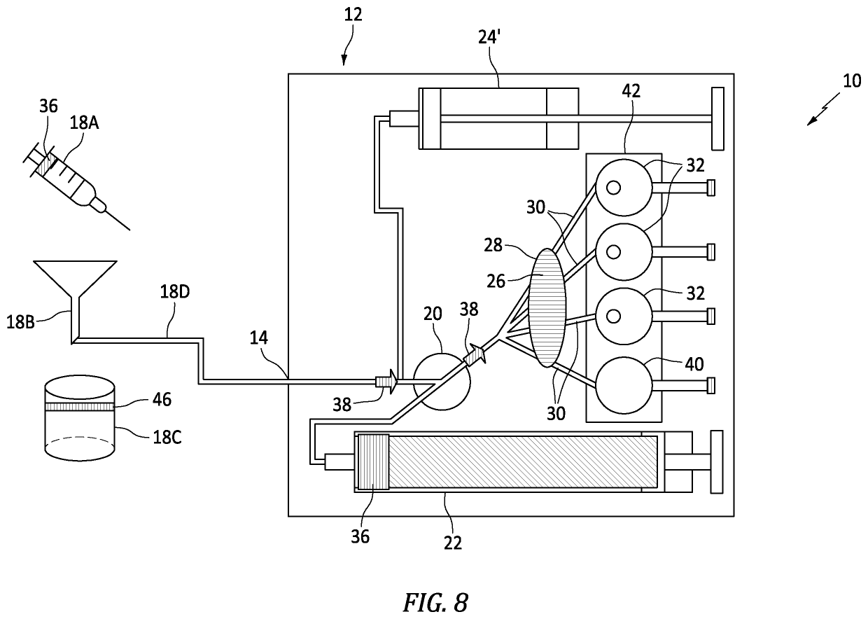

As shown in FIG. 7, in the fourth step, the patient or other user depresses the elution blister 24 which pushes the elution buffer 26 across the membrane 20 to elute all DNA and RNA off the membrane and into the capture reservoir 28. Fluid flow is controlled by the one-way valves 38, 38 which prevent fluid from flowing backwards from the reaction chambers 32, 32 into the membrane 20 and prevent any material from flowing backwards into the transfer/collection device(s) 18A, 18B, 18C and/or 18D. Once the RNA and DNA is in the capture reservoir 28, the reaction chambers 32, 32 are filled via capillary action which dissolves the reaction chemistry pre-stored in the reaction chambers as lyophilized beads. Depression of the elution blister also activates the device heating element 42 for heating the reaction chambers 32, 32 to the required temperature for the reaction. As shown in FIG. 8, in an alternative fourth step of the device 10, the patient or other use depresses the plunger of the elution syringe 24′ which pushes the elution buffer 26 across the membrane 20 to elute all DNA and RNA off the membrane and into the capture reservoir 28. Fluid flow is controlled by the one-way valves 38, 38 which prevent fluid from flowing backwards from the reaction chambers 32, 32 into the membrane 20 and prevent any material from flowing backwards into the transfer/collection devices 18A, 18B, 18C and/or 18D. Once the RNA and DNA is in the capture reservoir 28, the reaction chambers 32, 32 are filled via capillary action which dissolves the reaction chemistry pre-stored in the reaction chambers as lyophilized beads. Depression of the elution syringe 24′ also activates the device heater 42.

As shown in FIG. 5, in the fifth step of the device 10, heating of the reaction chambers 32, 32 initiates an amplification reaction where the target RNA/DNA is amplified, if present. Positive amplification is detected via user visualization (e.g., by color change as shown from blue to green, fluorescence, or a change in turbidity), or by digital image capture via an embedded CCD camera, photo-sensor or an external device such as a smart phone. Although in the illustrated device the heater 42 is an electric heater, a chemical heating or other type of heating mechanism that is currently known, or later becomes known, equally may be employed.

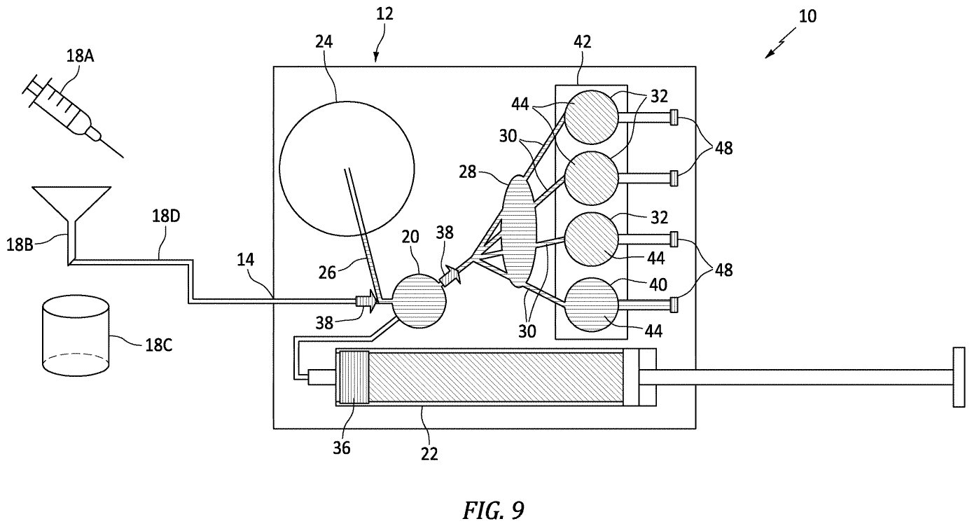

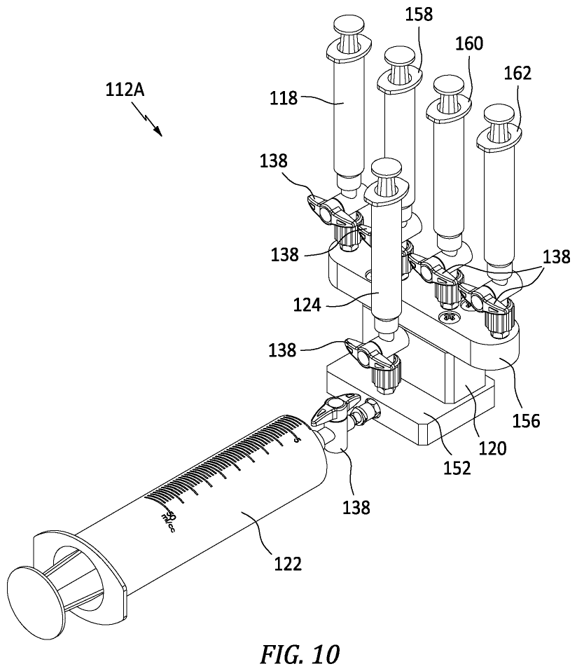

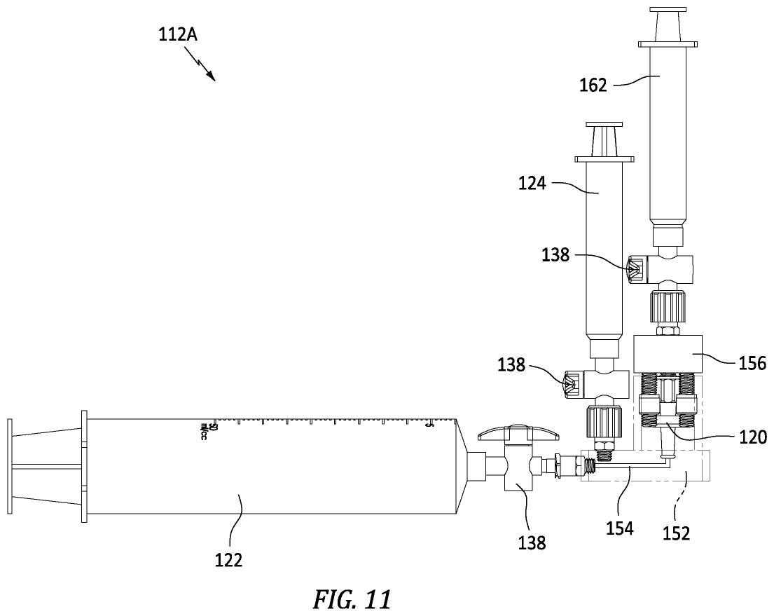

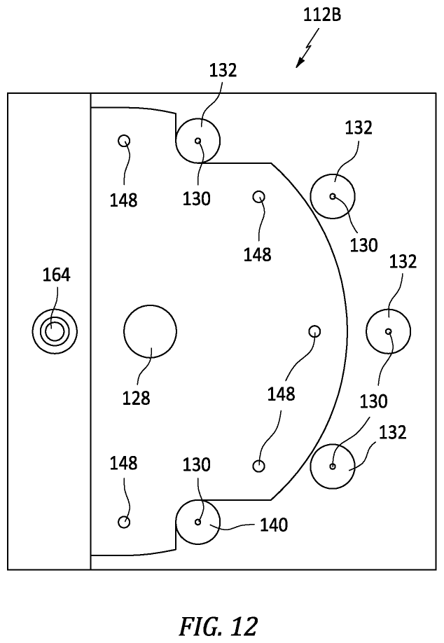

In FIGS. 10 and 11, another device embodying the present invention for sample collection and nucleic acid or RNA/DNA isolation and purification, is indicated generally by the reference numeral 112A. The device 112A is similar to the corresponding portions of the device 12 described above in connection with FIGS. 1 through 9, and therefore like reference numerals preceded by the numeral “1” are used to indicate like elements. The device 112A utilizes a manifold assembly containing a solid-state purification medium and fluid channels and acts as a structural chassis for a series of manually-actuated valves, fluid reservoirs and fluid actuation syringes that draw the sample and reagents from the fluid reservoirs, through the membrane and into waste and elution syringes. The elution fluid is then transferred to amplification devices for thermal incubation, as described above in connection with FIG. 9 or below in connection with FIGS. 12-17.

The device 112A comprises a waste syringe 122 coupled in fluid communication with an outlet side of a solid-state, purification membrane 120 through a waste draw valve 138 and a lower manifold 152. As shown in FIG. 11, the lower manifold 152 defines a fluid channel 154 therein extending in fluid communication between the outlet of the solid-state membrane 120 and the waste valve 138. An elution syringe 124 is connected in fluid communication to the fluid channel 154 through an elution valve 138. An upper manifold 156 is mounted on the lower manifold 152 on the inlet side of the solid-state membrane 120 and defines fluid channel(s) therein (not shown) in fluid communication with the inlet side of the solid-state membrane.

As shown in FIG. 10, a plurality of reservoirs and associated valves are mounted on top of the upper manifold 156. A sample-containing mixture reservoir 138 is mounted on the upper manifold 156 and is connected in fluid communication therewith through a respective sample valve 138. A first wash reservoir 158 is mounted on the upper manifold 156 adjacent to the sample reservoir 138 and is connected in fluid communication with the upper manifold through a respective first wash valve 138. A second wash reservoir 160 is mounted on the upper manifold 156 adjacent to the first wash reservoir 158 and is connected in fluid communication with the upper manifold through a respective second wash valve 138. An elution reservoir 162 is mounted on the upper manifold 156 adjacent to the second wash reservoir 160 and is connected in fluid communication with the upper manifold through a respective elution valve 138. In the illustrated embodiment, each reservoir is defined by a respective syringe where the reservoir is defined within the barrel of the syringe. Each syringe reservoir may include a Luer Lock or other type of connector on its distal end to releasably connect the syringe to, and disconnect the syringe from the respective valve or manifold. As may be recognized by those of ordinary skill in the pertinent art based on the teachings herein, the reservoirs may take the form of any of numerous different devices for storing, pumping, dispensing and/or otherwise releasing the respective liquid therein, that are currently known or that later become known, such as depressible blisters with frangible walls, or electrically actuated liquid dispensers. Also in the illustrated embodiment, the valves 138, 138 are manually-operable valves where each valve includes a respective knob that is manually turned or rotated to move the valve between a closed position preventing fluid flow through the valve and open position allowing fluid flow through the valve. As may be recognized by those of ordinary skill in the pertinent art, the valves 138, 138 may take the form of any numerous different types of valves, that may be operated or actuated in any of numerous different ways, that are currently known, or that later become known, including electrically or electronically operated or actuated valves.

In the operation of the device 112A, a sample mixture is loaded into the sample reservoir 118. Then the waste draw valve 138 is opened, and the sample reservoir valve 138 is opened to place the sample reservoir in fluid communication with the inlet side of the solid-state membrane 120, and the waste syringe 122 in fluid communication with the outlet side of the solid-state membrane 120. The user then retracts the plunger of the waste draw syringe 122 a first predetermined amount in order to pull or draw the sample mixture from the sample reservoir 118, through the upper manifold 156 and across the solid-state membrane 120. In the illustrated embodiment, the waste draw syringe 122 includes graduations or like indicia, and the predetermined amount is determined based on the movement of the plunger relative to the graduations or other indicia. However, as may be recognized by those of ordinary skill in the pertinent art based on the teachings herein, the predetermined amounts may be determined or dispensed with any of numerous different mechanisms, in accordance with any of numerous different ways, that are currently known, or that later become known, such as by electric or electronic metering. In one embodiment, such amount is equal to about 5 ml. After drawing the sample mixture out of the sample reservoir 118, the sample reservoir valve 138 is closed. Next, the first wash reservoir valve 138 is opened to place the first wash reservoir 158 in fluid communication with the inlet side of the solid-state membrane 120. The user then retracts the plunger of the waste draw syringe 122 a second predetermined amount in order to pull or draw the first wash from the first wash reservoir through the upper manifold 156 and across the solid-state membrane 120. In the illustrated embodiment, the second predetermined amount is about 5 ml. The user the closes the first wash reservoir valve 138 and opens the second wash reservoir valve 138 in order to place the second wash reservoir 160 in fluid communication with the inlet side of the solid-state membrane 120. The user then retracts the plunger of the waste draw syringe 122 a third predetermined amount in order to pull or draw the second wash from the second wash reservoir through the upper manifold 156 and across the solid-state membrane 120. In the illustrated embodiment, the third predetermined amount is about 10 ml. The user the closes the second wash reservoir valve 138 and closes the waste draw valve 138 to disconnect the waste draw syringe 122 from the solid-state membrane. The user then opens the elution valve 138 at the elution reservoir 162 to place the elution reservoir in fluid communication with the inlet side of the solid-state membrane 120, and opens the elution draw valve 138 at the elution draw syringe 124 in order to place the elution draw syringe in fluid communication with the outlet side of the solid-state membrane 120. The user then pulls the plunger of the elution draw syringe 124 in order to pull or draw the elution from the elution reservoir 162 across the solid-state membrane to elute the RNA and DNA therein, and capture the eluted RNA and DNA in the barrel of the elution draw syringe 124. The elution draw syringe 124 is disconnectable from its mount on the lower manifold 152, such as by a Luer connector, in order to transfer the elution therein to an amplification device for thermal incubation and amplification in reaction chamber(s) in the manner described above in connection with FIG. 9.

In FIGS. 12-17, another device embodying the present invention is indicated generally by the reference number 112B. The device 112B may be used in conjunction with the device 112A described above. As described above, the device 112A is for sample collection, and nucleic acid or RNA/DNA isolation and purification. The device 112B, on the other hand, is for amplification and detection of targeted isolated and purified nucleic acids or RNA/DNA from, for example, the device 112A. The device 112B is similar to the corresponding portions of the device 12 described above in connection with FIGS. 1 through 9, and therefore like reference numerals preceded by the numeral “1” are used to indicate like elements.

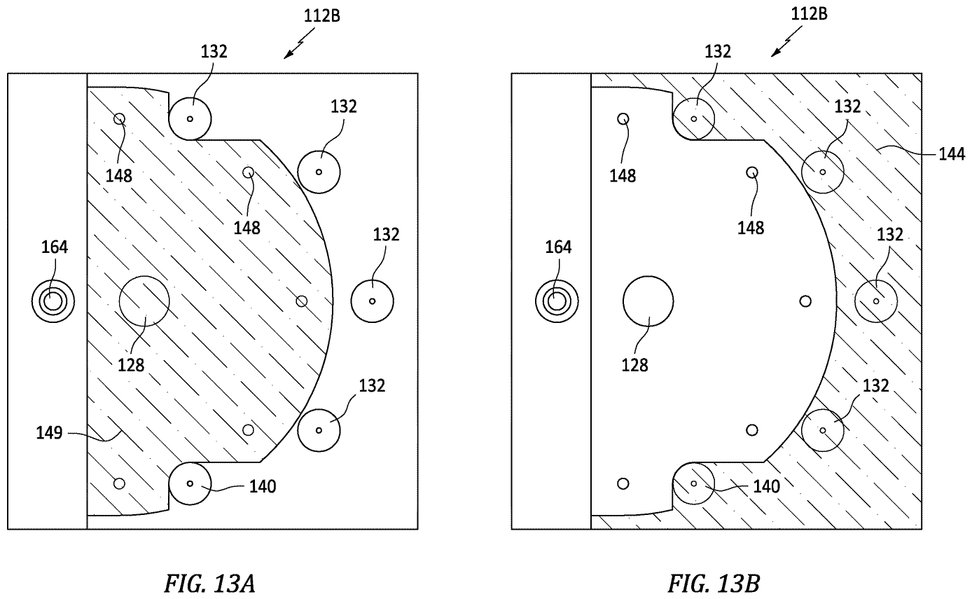

As shown in FIG. 12, the device 112B includes a fluid inlet 164 in fluid communication with a reservoir or premix chamber 128 which is in turn connected in fluid communication to a plurality of reaction/amplification chambers 132, 132 through respective capillary conduits 130, 130. Each reaction/amplification chamber 132, 132 includes a respective reaction/amplification chamber vent 148, 148 in fluid communication therewith for venting the respective chamber to the ambient atmosphere. In FIG. 13A, a single vent membrane component 148 covers the plurality of vented ports 148, 148 in fluid communication with respective reaction chambers 132, 132 and the negative control chamber 140 for venting the chambers therethrough. The vent membrane 148 permits air or other gas to flow therethrough, but prevents or substantially prevents liquid flow therethrough.

As shown in FIG. 13B, a one-piece optically clear material 144 covers each reaction chamber 132, 132 and the negative control chamber 140 to thereby form a transparent/translucent viewing window over each reaction chamber or negative control chamber for viewing the interior of each chamber therethrough. The transparent or translucent window 144 is formed of an optically clear material of a type known to those of ordinary skill in the pertinent art which covers each reaction/amplification chamber 132, 132 and the negative control chamber 140 to allow viewing of the interior of the chamber therethrough. As may be recognized by those of ordinary skill in the pertinent art based on the teachings herein, the device 112B may include a single vent membrane component 148 and a single viewing window components 144 for all reaction chambers 132, 132, as shown, or may include any desired numbers of such components.

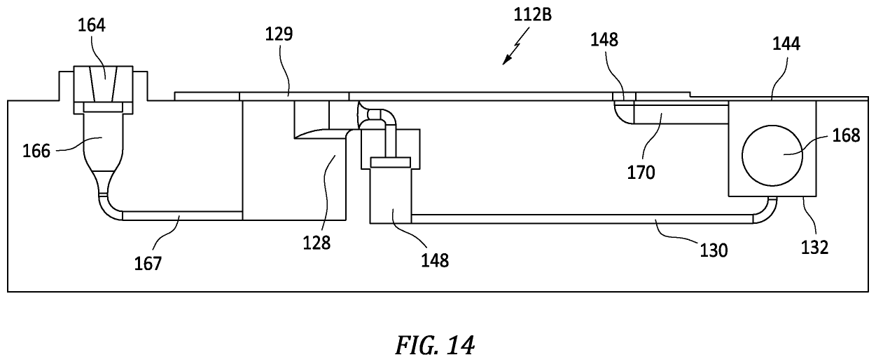

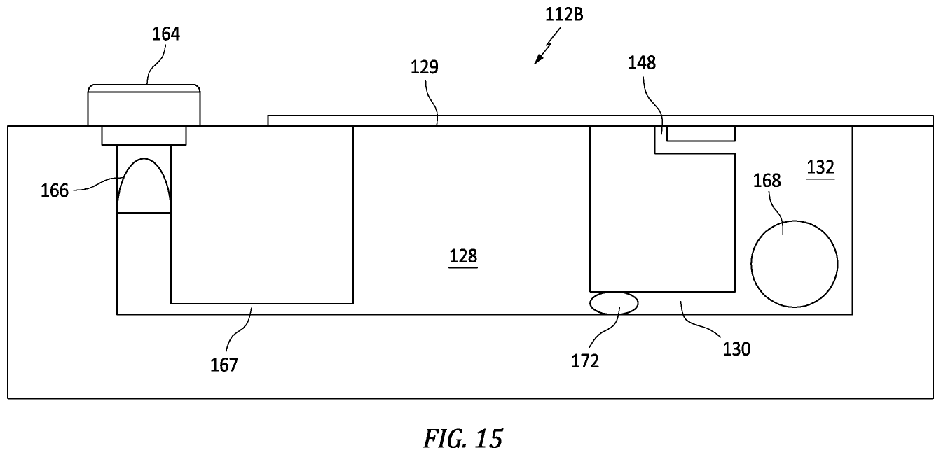

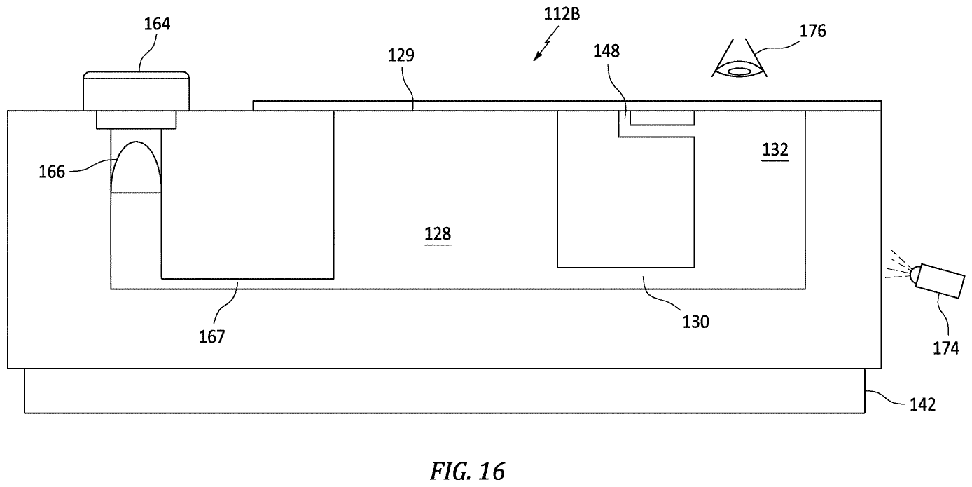

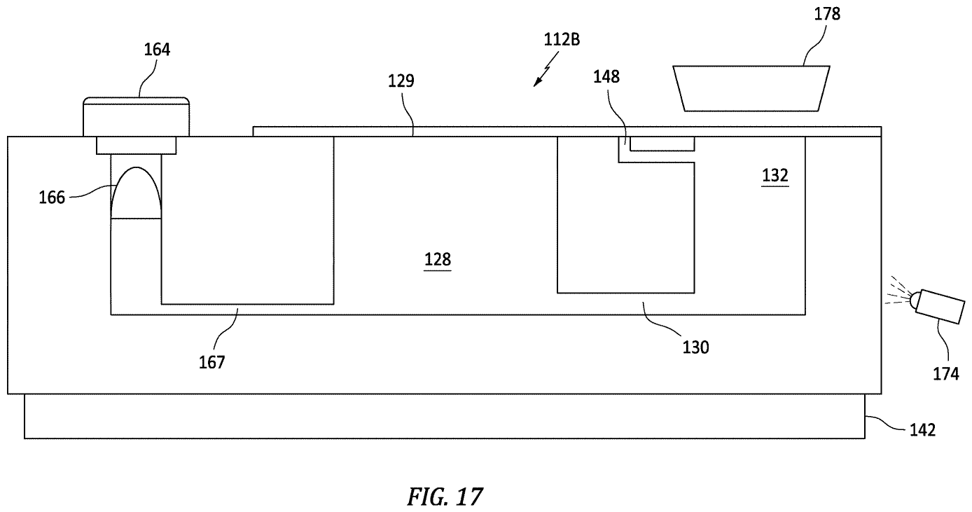

As shown in FIGS. 14 and 15, the fluid inlet 164 is in fluid communication with an inlet check valve 166 which is in turn connected through a fluid-flow path 167 to the reservoir/premix chamber 128. The fluid inlet 164 is configured to receive the distal end of the elution syringe reservoir 162 for receiving the elution therefrom, as described above in connection with FIG. 10. The reservoir/premix chamber 128 includes a vent 129 for venting the chamber during filling thereof. As shown in FIG. 14, a pressure relief valve 148 is coupled in fluid communication between the reaction/premix chamber 128 and the plurality of capillary conduits 130, 130 which are, in turn, connected in fluid communication with the respective reaction/amplification chambers 132 132. As shown in FIG. 15, each capillary conduit 130 includes therein a respective wax or other meltable pellet 172 that is configured to prevent the transfer of liquid from the reservoir/premix chamber 128 through the conduit at room temperature (or temperatures below the incubating temperature). The wax pellet 172 is configured so that it does not melt at room temperature, but rather melts at a temperature near, or approaching the incubation temperature for the amplification reaction. One advantage of this feature is that it gives the solution more time to homogenize and distribute DNA/RNA more consistently to each reaction chamber 132, 132. When the premix chamber 128 is heated by the heater 142 (FIGS. 16 and 17) the rate of diffusion increases and thus mixes the contents. Time and temperature (i.e., at or near the incubation temperature) facilitate such mixing. It should be noted that when the wax 172 melts, capillary action draws the solution into the conduits 130, 130 and reaction chambers 132, 132, while the vent 129 allows air to enter back into the premix chamber 128 in order to backfill the volume inside of the premix chamber as fluid is displaced therefrom. Each reaction/amplification chamber 132, 132 preferably contains a lyophilized master mix bead or reagent mix 168 therein, and a respective portion of the transparent or translucent viewing window 144 overlies each such chamber and the negative control chamber 140 for visual observation of the interior of each chamber therethrough following the reaction/amplification. A vent channel 170 extends between an upper end or region of each reaction/amplification chamber 132, 132 and a respective reaction/amplification chamber vent 148 for venting the chamber therethrough.

In the operation of the device 112B, the check valve 166 at the fluid inlet 164 passively closes once the reservoir/premix chamber 128 is filled with elution fluid 126. Upon entry of the elution fluid 126 into the device 112B, the reservoir/pre-mix chamber 128 fills completely as air escapes from the reservoir/premix chamber vent 148 completing the premix process and resulting in a substantially uniform concentration of DNA/RNA throughout the fluid volume in the reservoir/premix chamber. The fluid pressure inside the reservoir/premix chamber 128 rises as fluid is added thereto. Once the reservoir/premix chamber 128 is filled (or reaches a predetermined level of fill), the pressure inside the reservoir/premix chamber 128 exceeds the relief pressure of the pre-mix chamber pressure relief valve 148 which, in turn, allows the substantially homogeneous liquid in the reservoir/premix chamber 128 to flow into each of five amplification/reaction chambers 132, 132 and negative control chamber 140. In the amplification/reaction chambers 132, 132, the substantially homogenous liquid dissolves the reagent master mix 168 while air is vented out through the respective reaction/amplification chamber vents 148, 148.

The device 112B is then incubated by the heating unit or element 142, inducing target RNA/DNA amplification in the reaction/amplification chambers. If desired, each reaction/amplification chamber 132, 132 can contain a unique master mix 168 for amplification of different targets, if desired. As shown in FIG. 16, the heater 142, which may be, for example, an electric or chemical heater, is in thermal communication with the reaction chambers 132, 132 and negative control chamber 140. Following actuation of the heater 142 to a predetermined incubation temperature, the lyo-bead or other reaction chemistry 168 is dissolved and the wax pellet 172 is melted away. After incubating the filled reaction chambers 132, 132 at a predetermined incubation temperature and time period, and the resulting amplification of the RNA/DNA, an excitation LED or other light source 174 causes the positive indicator to fluoresce, such that when viewed by an eye 176 of an operator, the fluorescence indicates positive detection of target DNA/RNA. Calcein, also known as fluorexon, or fluorescein complex, is a fluorescent dye with excitation and emission wavelengths of 495 nm and 515 nm, respectively. Calcein is commonly used in biological applications as a marker for detection of certain biological agents. In the device 112B, calcein is a component in the lyo-bead master mix 168. In the lyo-bead form, the calcein is not capable of emitting the excitation wavelength (510 nm). During the amplification/incubation, if the target molecule is present in the elution within the respective reaction chamber 132, the calcein contained within the master mix (i.e., in the lyo-bead) will undergo a chemical change that permits emission of the excitation light enabling the operator to view a fluorescent glow, indicating the presence of the target molecule in the sample, i.e. a positive detection of the target. During incubation, when the target molecule is not present in the reaction chamber 132, 132, the chemical change is not induced and the reaction will not glow, indicating the target molecule is not present in the sample, i.e., a negative result.

As shown in FIG. 17, in lieu of the human readable embodiment of FIG. 16, the device 112B includes a photo-sensor 178 configured to output a digital binary signal, indicating either a positive or negative result, to a user interface that communicates the results to a user. One advantage of this embodiment is the ease of use and fitness for use by a less skilled operator as may be necessary for some point-of-care (“POC”) applications. As in FIG. 16 above, at the stage shown in FIG. 17, the wax pellet 172 is melted away. In addition, the lyo-bead 168 will have been dissolved by the time amplification has occurred, when the results are read. The photo-sensor 178 measures the color and intensity of the emission wavelength in a respective reaction chamber 132, or in plural reaction chambers 132, 132. When the 515 nm power rises above a predetermined set threshold, the result is deemed positive. If, on the other hand, the 515 nm power does not rise above the predetermined set threshold, the result is deemed negative. In either case, the photo-sensor 178 transmits a signal indicative of either a positive result or a negative result to a user interface, such as a display or smart phone, that communicates the results to a user. As may be recognized by those of ordinary skill in the pertinent art based on the teachings herein, the device 112B may include one photo-sensor or other sensor for all chambers, or plural sensors for plural chambers, where each sensor senses the emission wavelengths within the interior(s) of one or more chambers.

As may be recognized by those of ordinary skill in the pertinent art based on the teachings herein, numerous changes, improvements, modifications, additions and deletions may be made to the above-described and other embodiments of the present invention without departing from the scope of the invention. For example, the microfluidic devices or components thereof, and the methods of operation or use, or aspects thereof, may be the same as or similar to any of the microfluidic devices or components thereof, and methods or aspects, disclosed in the following co-pending patent applications, which are assigned to the assignee of the present invention and are hereby incorporated by reference in their entireties as part of the present disclosure: (i) U.S. patent application Ser. No. 17/647,828, filed Jan. 12, 2022, entitled “Device And Method For Detecting Nucleic Acids In Biological Samples,” and (ii) U.S. patent application Ser. No. 17/941,816, filed Sep. 9, 2022, entitled “Device And Method For Detecting Nucleic Acids In Biological Samples.” In addition, the device may include fewer parts, or additional parts than those illustrated and/or described herein. For example, the device may include only one pump for pumping the sample mixture, and dodecanol and air, any wash solutions and any eluents across one or more solid-state membranes. Alternatively, the device may include multiple pumps for performing such functions. Alternatively, the device may include multiple solid-state membranes or other filtration mechanisms, including membranes mounted in series. Still further, the device may include plural capture reservoirs, or in other cases, the capture reservoir may be eliminated. In other embodiments, the conduit(s) running between the capture reservoir and the reaction chamber(s) and/or negative control chamber(s) need not be capillary or operate by capillary flow action. For example, flow through the conduits may be achieved via pressure differential, such as by the pumping that fills the capture reservoir. In addition, the heating element need to operate in an on/off scenario, but rather may operate by thermo or thermal cycling, such as for PCR or other non-lamp methods/applications. The solid-state membrane also may take the form of any device that is currently known, or that later becomes known for capturing thereon and releasing nucleic acids, RNA and/or DNA, such as glass beads, including, for example, boro-silicate glass beads. Accordingly, the components of the device(s) and the methods of operating or using the device(s), may take any of numerous different forms or configurations, and may be made of or use any of numerous materials, that are currently known or later become known, and features or aspects may be added or removed, without departing from the scope of the invention. This detailed description of embodiments is therefore to be taken in an illustrative as opposed to a limiting sense.

What is claimed is:

1. A device comprising:
a sample port or chamber for receiving therein a sample-containing mixture containing therein a biological sample;
a solid-state membrane in fluid communication with the sample port or chamber and configured to receive the sample-containing mixture therefrom, and allow the sample-containing mixture to pass across the membrane and capture nucleic acids in the biological sample on the membrane;
a first pump in fluid communication with the solid-state membrane and a waste chamber in fluid communication with the pump, wherein actuation of the first pump causes the sample-containing mixture to flow across the solid-state membrane and into the waste chamber;
an eluent chamber configured to contain or containing an eluent therein;
an eluent reservoir in fluid communication with the solid-state membrane; and
a second pump in fluid communication with the solid-state membrane and the eluent chamber, wherein actuation of the second pump causes the eluent to flow from the eluent chamber across the solid-state membrane, elute captured nucleic acids from the solid-state membrane, and flow with the captured nucleic acids into the eluent reservoir.

2. A device as defined in claim 1, wherein the first pump is a syringe containing a barrel and a plunger received within the barrel, the barrel defines the waste chamber therein, and movement of the plunger draws or pulls the sample-containing mixture across the solid-state membrane and into waste chamber of the barrel.

3. A device as defined in claim 2, wherein the second pump is movable between a non-actuated position and an actuated position, the eluent chamber includes a frangible or breakable wall that is breakable by movement of the second pump between the non-actuated position and the actuated position to pump eluent from the eluent chamber across the solid-state membrane and into the eluent reservoir.

4. A device as defined in claim 3, wherein the second pump is a plunger, movement of the plunger from the non-actuated position to the actuated position breaks the frangible or breakable wall of the eluent chamber and pushes the eluent across the solid-state membrane and into the eluent reservoir.

5. A device as defined in claim 2, wherein the second pump is a syringe including a barrel and a plunger received within the barrel, and movement of the plunger pushes the eluent across the solid-state membrane and into the eluent reservoir.

6. A device as defined in claim 1, further comprising a first one-way valve in fluid communication between the sample port or chamber and the solid-state membrane and configured to allow the sample-containing mixture to flow in the direction from the sample port or chamber to the solid-state membrane.

7. A device as defined in claim 1, further comprising a second valve in fluid communication between the solid-state membrane and the eluent reservoir and configured to allow fluid flow in the direction from the solid-state membrane into the eluent reservoir.

8. A device as defined in claim 1, further comprising at least one reaction chamber and at least one conduit in fluid communication between the eluent reservoir and the reaction chamber,

9. A device as defined in claim 8, wherein the at least one conduit is a capillary conduit configured to allow the eluent with captured nucleic acid to flow by capillary action through the capillary conduit and into the reaction chamber.

10. A device as defined in claim 1, further comprising a plurality of reaction chambers and conduits in fluid communication between the eluent reservoir and each reaction chamber, wherein each conduit is configured to allow the eluent with captured nucleic acid to flow through the conduit and into the respective reaction chamber.

11. A device as defined in claim 8, further comprising a valve in fluid communication between the reaction chamber and the ambient atmosphere, wherein the valve allows gas to flow from the reaction chamber into the ambient atmosphere, but prevents liquid flow therethrough.

12. A device as defined in claim 8, further comprising a heating element in thermal communication with the reaction chamber, wherein the heating element defines a first condition where the heating element heats the reaction chamber to an incubation temperature, and a second condition where the heating element does not heat the reaction chamber to the incubation temperature, and the heating element is configured to transition from the first condition to the second condition upon or following actuation of the second pump.

13. A device as defined in claim 8, further comprising an optical sensor configured to measure at least one of emission wavelength or intensity within the reaction chamber to detect at least one of a positive detection of a targeted nucleic acid or a negative detection of a targeted nucleic acid, and to transmit a signal to a user interface indicative thereof.

14. A device comprising:
first means for receiving therein a sample-containing mixture containing therein a biological sample;
second means in fluid communication with the first means for receiving the sample-containing mixture therefrom, for allowing the sample-containing mixture to pass across the second means, and for capturing nucleic acids in the biological sample on the second means;
third means for receiving and holding the sample-containing mixture after passing across the second means;
fourth means in fluid communication with the second means and the third means for pumping the sample-containing mixture across the second means and into the third means;
fifth means for containing an eluent therein and for allowing the eluent to flow across the second means after the sample-containing mixture passes across the second means, and for removing from the second means captured nucleic acids from the biological sample with the eluent;
sixth means in fluid communication with the solid-state membrane for receiving and collecting the eluent with captured nucleic acids from the biological sample therein; and
seventh means in fluid communication with the second means and the fifth means for pumping the eluent to flow from the fifth means across the second means, elute captured nucleic acids from the second means, and flow with the captured nucleic acids into the sixth means.

15. A device as defined in claim 14, wherein the first means is a sample port or chamber, the second means is a solid-state membrane, the third means is a waste chamber, the fourth means is a first pump, the fifth means is an eluent chamber, the sixth means is an eluent reservoir, and the seventh means is a second pump.

16. A formulation for collecting a biological sample of saliva or nasal fluid and capturing nucleic acids in the collected biological sample on a solid-state membrane, comprising: one or more non-toxic chaotropic agents; ethanol; and/or coloring and/or flavoring agents, wherein the formulation is receivable within an oral cavity or nasal cavity to collect the biological sample of saliva or nasal fluid therefrom, and the one or more non-toxic chaotropic agents and ethanol at least one of lyse the cells of the biological sample or facilitate the binding of nucleic acids in the biological sample to the solid-state membrane.

17. A formulation as defined in claim 16, comprising about 0.1% to about 40% w/v non-toxic chaotropic agents and about 5% to about 30% w/v ethanol.

18. A formulation as defined in claim 17, wherein the non-toxic chaotropic agents are selected from the group including the following individually or in any combination thereof: (i) about 5% to about 30% w/v urea; about 0.1% to about 3% w/v sodium lauryl sulfate; and about 2% to about 40% w/v ammonium trichloroacetate.

19. A formulation as defined in claim 16, in combination with a long-chain fatty alcohol wash configured to flow over the solid-state membrane following the formulation to substantially eliminate any residual ethanol of the formulation on the solid-state membrane.

20. A combination as defined in claim 19, further comprising a transfer device containing in a first portion thereof the formulation and containing in a second portion thereof the long-chain fatty alcohol wash, wherein the first portion thereof is receivable within an oral cavity or nasal cavity for collecting saliva or nasal fluid, and the transfer device is configured to transfer or dispense the formulation and collected biological sample to the solid-state membrane and then transfer or dispense the long-chain fatty alcohol wash to the solid-state membrane.

21. A combination as defined in claim 20, wherein the transfer device is a syringe comprising on a distal end thereof a gauze or other absorbent material that is receivable in the oral cavity or nasal cavity and configured to absorb the saliva or nasal fluid therein, wherein the syringe defines a first chamber or portion of a chamber in fluid communication with the gauze or other absorbent material and containing therein the formulation, and a second chamber or portion of a chamber located on an opposite side of the first chamber or portion thereof relative to the gauze or other absorbent material and containing therein the long-chain fatty alcohol wash, wherein actuation of the syringe causes the formulation to flow through the gauze or other absorbent material and collect therein biological sample and to dispense from the syringe a mixture of the formulation and biological sample, and further actuation of the syringe causes the long-chain fatty alcohol wash to be dispensed from the syringe following dispensing of the formulation and biological sample mixture.

22. A combination as defined in claim 21, wherein the syringe includes a barrel defining the first and second chambers, and includes a frangible or breakable wall separating the first and second chambers, and the syringe further comprises a plunger received within the barrel, whereupon actuation of the plunger breaks the frangible or breakable wall and dispenses the formulation and then the long-chain fatty alcohol wash from the syringe.
